(19) United States
(12) Patent Application Publication
(10) Pub. No.: US 2002/0042601 A1
(43) Pub. Date: Apr. 11, 2002

(54) RESILIENT ATTACHMENT MATERIALS FOR PERSONAL CARE PRODUCTS

(76) Inventors: TIMOTHY RAY MARTIN, ALPHARETTA, GA (US); RICHARD JOHN SCHMIDT, ROSWELL, GA (US)

Correspondence Address: JAMES B. ROBINSON, KIMBERLY-CLARK WORLDWIDE, INC., 401 NORTH LAKE STREET, NEENAH, WI, 54956

(21) Appl. No.: 09/390,100

(22) Filed: Sep. 3, 1999

Related U.S. Application Data
(60) Provisional application No. 60/102,025, filed on Sep. 28, 1998.

(51) Int. Cl. A61F 13/15; A44B 1/04; A44B 11/25; A44B 17/00; A44B 18/00

(52) U.S. Cl. 604/391; 604/387; 24/442

(57) ABSTRACT

There is provided a hook and loop attachment means made up of a hook portion and a loop portion where the loop and/or the hook portion is convex and is underlain by a resilient material or hollow area. The hook and loop attachment means is useful in the attachment of personal care products.

This application claims priority from U.S. Provisional Patent Application 60/102,025, filed Sep. 28, 1998.

### FIELD OF THE INVENTION

The present invention relates to a structure in an article for personal care like diapers, training pants, absorbent underpants, adult incontinence products, swim wear, bandages and feminine hygiene products.

### BACKGROUND OF THE INVENTION

Personal care articles include such items as diapers, training pants, feminine hygiene products such as sanitary napkins, incontinence garments and devices, swim wear, bandages and the like. The most basic design of such articles usually includes a bodyside liner, an outercover and an absorbent core disposed between the bodyside liner and the outercover.

Various attachment means have been used to keep the personal care product in place on the wearer. Tape, hook and loop systems and slip on garment designs requiring no separate attachment devices, for example, have been used. Each of these means has its own idiosyncratic deficiency, however. Tape, for example, will sometimes not adhere properly if there is lotion or other contaminants in the area of attachment, also known as the “landing zone”. Hook and loop fasteners sometimes fail due to lateral forces, for example, and slip on designs cannot be easily opened and closed to check for insults. There remains a need for an attachment means, which is not easily affected by contaminants, resists lateral forces well and which may be detached and reattached easily a number of times.

It is an object of this invention, therefore, to provide an attachment means which can be reattachably detached, is resistant to contaminants in the landing zone, and which holds up well to peel and shear forces. It is a further object of this invention to provide personal care products incorporating such a superior attachment means.

### SUMMARY OF THE INVENTION

The objects of the invention are achieved by a hook and loop attachment means, which is made up of a hook portion and a loop portion where at least one of the portions is convex and is underlain by a resilient material or hollow area. The hook and loop attachment means is useful in the attachment of personal care products.

### BRIEF DESCRIPTION OF THE DRAWINGS

FIG. 1 is a photograph of the convex hook component of this invention showing individual hooks as well as the curvature of the entire hook component.

FIG. 2 is a photograph showing the individual hooks of a hook and loop fastener wherein the hooks face in different directions.

FIG. 3 is a drawing of a typical personal care product, in this case a diaper, showing the front, back and the hook and loop components in their relative positions.

FIG. 4 shows a cross section view of a hook component underlain by a resilient material which is in turn attached to, or an integral part of, a backing material which may be a personal care product.

FIG. 5 shows a variation of FIG. 4 wherein the resilient underlaying material is absent and the hook component is folded in such a manner as to cause it to rise out of the flat plane of the backing.

FIG. 6 shows another variation of FIG. 4 wherein there are a multiciplicities of convexities in the hook material. These may be formed by placing an underlayment below the hooks or by folding.

FIG. 7 is a picture showing a hollow underlayment, which may be used in the practice of the invention, in a position of attachment.

FIG. 8 is a picture showing a resilient underlayment, which may be used in the practice of the invention, in a position of attachment.

FIG. 9 is a picture showing a resilient underlayment, which may be used in the practice of the invention, in a position wherein the material is being peeled.

### DEFINITIONS

“Disposable” includes being disposed of after use and not intended to be washed and reused.

“Front” and “back” are used throughout this description to designate relationships relative to the garment itself, rather than to suggest any position the garment assumes when it is positioned on a wearer.

“Layer” when used in the singular can have the dual meaning of a single element or a plurality of elements.

By the term “similar hook” what is meant is a hook which uses essentially the same hook as the inventive material but in which the hook component is flat and not convex. According to Webster's New Collegiate Dictionary (1980), “similar” means 1) having characteristics in common; strictly comparable, 2) alike in substance or essentials; corresponding. Using this commonly accepted meaning of the word similar, this term means that all other conditions are essentially the same except for the conditions mentioned.

As used herein the term “polymer” generally includes but is not limited to, homopolymers, copolymers, such as for example, block, graft, random and alternating copolymers, terpolymers, etc. and blends and modifications thereof. Furthermore, unless otherwise specifically limited, the term “polymer” shall include all possible geometrical configurations of the molecule. These configurations include, but are not limited to isotactic, syndiotactic and random symmetries.

As used herein the term “nonwoven fabric or web” means a web having a structure of individual fibers or threads, which are interlaid, but not in an identifiable manner as in a knitted fabric. Nonwoven fabrics or webs have been formed from many processes such as for example, meltblowing processes, spunbonding processes, and bonded carded web processes. The basis weight of nonwoven fabrics is usually expressed in ounces of material per square yard (osy) or grams per square meter (gsm) and the fiber diameters useful are usually expressed in microns. (Note that to convert from osy to gsm, multiply osy by 33.91).

“Spunbonded fibers” refers to small diameter fibers that are formed by extruding molten thermoplastic material as filaments from a plurality of fine capillaries of a spinneret. Such a process is disclosed in, for example, U.S. Pat. No. 4,340,563 to Appel et al., and U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartman, and U.S. Pat. No. 3,542,615 to Dobo et al. The fibers may also have shapes such as those described in U.S. Pat. No. 5,277,976 to Hogle et al., U.S. Pat. Nos. 5,466,410 to Hills and 5,069,970 and 5,057,368 to Largman et al., which describe fibers with unconventional shapes.

“Conjugate fibers” refers to fibers which have been formed from at least two polymers arranged in substantially constantly positioned distinct zones across the cross-section of the fibers and which extend continuously along the length of the fibers. Conjugate fibers are taught in U.S. Pat. No. 5,108,820 to Kaneko et al., U.S. Pat. No. 5,336,552 to Strack et al., and U.S. Pat. No. 5,382,400 to Pike et al.

“Bonded carded web” refers to webs that are made from staple fibers which are sent through a combing or carding unit, which separates or breaks apart and aligns the staple fibers in the machine direction to form a generally machine direction-oriented fibrous nonwoven web. Such fibers are usually purchased in bales, which are placed in an opener/blender, or picker, which separates the fibers prior to the carding unit. Once the web is formed, it then is bonded by one or more of several known bonding methods. One such bonding method is powder bonding, wherein a powdered adhesive is distributed through the web and then activated, usually by heating the web and adhesive with hot air.

“Personal care product” means diapers, training pants, absorbent underpants, adult incontinence products, swimwear, bandages and feminine hygiene products.

“Feminine hygiene products” means sanitary napkins or pads, and panty-liners.

“Shear force” refers to forces acting generally parallel to the plane of the substrate of the hook and loop fastening system.

“Peel forces” refers to forces acting generally perpendicular to the plane of the substrate of the hook and loop fastening system.

### TEST METHODS AND MATERIALS

Peel Test: The peel force value measures the force needed to peel apart a hook and loop fastening system and can be determined in accordance with standard procedure ASTM D5170, approved Sept. 15, 1991 and published November 1991; with the following particulars. The loop material to be tested is cut into a rectangle, 76 mm (3 inch) by 152 mm (6 inch) with the longer dimension in the cross-machine direction. The loop material is placed under the clamping plate of a rolldown machine. The hook material is placed on top of the loop material and attached by the rolldown machine using a 2 kg roller. A suitable rolldown machine is part number HR-100 available from Chemsultants International, of Mentor, Ohio. During the engagement of the fastener components, the roller is rolled over the test specimen through one cycle in the direction of the cross-wise “width” of the sample. In addition, the initial peel by hand to “raise the loops” is omitted. After the hook and loop are properly attached, the combination is placed in the testing apparatus, an Instron Model 2712-004 tensile tester with 102 mm (4 inch) rubberized grip faces (Instron Corporation, Canton Mass. 02021). The hook base is inserted in the upper grip and the loop in the lower in such a manner that the movement of the grips away from each other will result in the peeling apart of the two materials. Slack is removed and the machine is started. The tester is set with a crosshead speed of 500 mm/min. and a gage length of 76 mm. Measurements are begun at 10 mm and end at 46 mm and are in grams. The reported value of a peel test result is a peal load value employing MTS TESTWORKS software with a peak criteria of 2%. Additionally, the peel force value is normalized to be stated in terms of force per unit length of the “width” dimension of the fastener component on the test specimen, such as grams per inch or grams per centimeter. The MTS TESTWORKS software is available from MTS Systems Corporation, a business having offices in Eden Prairie, Minn.

Shear Test: The shear force value measures how well the hook and loop stay engaged against in-plane shear force and can be determined in accordance with the standard procedure ASTM D-5169, approved Sep. 15, 1991 and published November 1991 with the following particulars. The materials are prepared in the same manner as in the peel test except that non-printed loop material only is cut in to a size of 25 mm (1 inch) in the machine direction by 152 mm (6 inch) in the cross machine direction. After the rolldown machine has engaged the hook and loop materials to each other as described above, they are removed and placed in the Instron Model 2712-004 tensile tester, hooks in the upper grip, loops in the lower in such a manner that the movement of the grips away from each other will not result in the peeling apart of the two materials. Slack is removed and the machine is started. The tester is set with a crosshead speed of 500 mm/min. and a gage length of 76 mm. Measurements are begun at 10 mm and end at 46 mm and are in grams. The shear force value is normalized to be stated in terms of force per unit area of the test specimen, such as grams-force per inch² (or gmf/cm²).

Material caliper (thickness): The caliper of a material is a measure of thickness and is measured at 0.05 psi with a Starret-type bulk tester, in units of millimeters.

Density: The density of the materials is calculated by dividing the weight per unit area of a sample in grams per square meter (gsm) by the bulk of the sample in millimeters (mm) at 68.9 Pascals and multiplying the result by 0.001 to convert the value to grams per cubic centimeter (g/cc). A total of three samples would be evaluated and averaged for the density values.

### DETAILED DESCRIPTION OF THE INVENTION

Personal care products include such items as diapers, training pants, feminine hygiene products such as sanitary napkins, panty-liners and tampons, incontinence garments and devices, swim wear, bandages and the like. The most basic design of such articles typically includes a bodyside liner, an outercover and an absorbent core disposed between the bodyside liner and the outercover. Also necessary is some sort of attachment means so that the product stays in place. Optional layers include fluid transfer and distribution layers.

The bodyside layer is sometimes referred to as a bodyside liner or topsheet. In the thickness direction of the article, the liner material is the layer against the wearer's skin and so the first layer in contact with liquid or other exudate from the wearer. The liner further serves to isolate the wearer's skin from the liquids held in an absorbent structure and should be compliant, soft feeling and non-irritating.

The bodyside liner can be surface treated with a selected amount of surfactant or otherwise processed to impart the desired level of wettability and hydrophilicity. If a surfactant is used, it can be an internal additive or applied to the layer by any conventional means, such as spraying, brush coating and the like, prior to the deposition of the next layer.

The fluid retention or absorbent core layer must absorb liquid from the adjacent bodyside layer in a controlled manner such that liquid may be stored away from contact with the body. Retention materials generally comprise binder, synthetic fibers and natural fibers. While any of the layers of a personal care product may optionally contain a superabsorbent, the fluid retention layer is the most logical layer to contain such a component.

The garment side liner layer, also referred to as a backsheet or outer cover is the farthest layer from the wearer. The outer cover functions to prevent body exudates contained in an absorbent structure from wetting or soiling the wearer's clothing, bedding, or other materials contacting the personal care product. The outer cover has traditionally been formed of a thin thermoplastic film, such as polyethylene film, which is substantially impermeable to liquid but may optionally be composed of a vapor or gas permeable, microporous “breathable” material, that is permeable to vapors or gas yet substantially impermeable to liquid.

The optional fluid transfer layer, also referred to as a surge layer, is most typically interposed between and in intimate, liquid communicating contact with the bodyside liner and another layer such as a fluid distribution or absorbent core layer. The fluid transfer or surge layer allows fluid movement through itself generally in the Z-direction, i.e. away from the bodyside and towards the garment side.

An optional distribution layer may be interposed above (toward a wearer) the fluid retention layer and must be capable of moving fluid from the point of initial deposition to where storage is desired. The fluid distribution layer, therefore, in addition to allowing fluid movement through itself in the Z-direction, also moves fluid in the X and Y directions. Distribution must take place at an acceptable rate such that the target insult area, generally the crotch area, is ready for the next insult. The time between insults can range from just a few minutes to hours, generally depending on the age of the wearer and the personal care product in question.

The breathable outercover discussed above is an example of the approach, which has been used previously to increase the ability of air to enter the product. The breathable outercover allows air through, and, in bench testing, can provide good air flow rates as measured by conventional ASTM permeability testing. While a breathable outercover will permit air to flow through, in actual use there is no way to guarantee the flow of air through the product and all the way to the skin, since this method is entirely passive. The dynamic air flow provision remedies this by avoiding the exclusive reliance on passive air entry and forcing or drawing air into the product into the region between the skin and the product.

Various attachment means have been used to keep the personal care product in place on the wearer. Adhesive tape, mechanical systems and slip-on garment designs requiring no separate attachment devices, for example, have been used. Oils and powders less easily contaminate attachment means such as mechanical fastening systems than they contaminate adhesive tape fastening systems. Additionally, mechanical fastening systems may be easily reused. All of these features provide advantages when applied to a disposable diaper intended for use on an infant, for example. Also, a refastenable mechanical fastening system provides the advantage the infant may be checked to see if soiling of the disposable diaper has occurred during the wearing period.

Mechanical fastening systems typically employ two components—a male (hook) component and a female (loop) component. The hook component usually includes a plurality of semi-rigid, hook-shaped elements anchored or connected to a base material. The loop component generally includes a backing material from which a plurality of upstanding loops project. The hook-shaped elements of the hook component are designed to engage the loops of the loop material, thereby forming mechanical bonds between the hook and loop elements of the two components.

The loop component of the fastening system typically is placed on the body of the garment and the hook component of the fastening system is placed on a strap or “ear”, which may be elastic. For example, U.S. Pat. No. 5,664,302 describes hook and loop fasteners wherein the loop material is underlain by a substrate, which is a sheet of elastic material. Hook fastening materials and methods of making such hook fastening materials are disclosed in U.S. Pat. Nos. 5,326,415, 5,318,741, 5,230,851, 5,058,247 and 5,116,567. Examples of hooks for hook and loop fasteners may also be found in U.S. Pat. No. 4,984,339, assigned to Velcro Industries BV of the Netherlands. An example of a suitable loop material may be found in U.S. Pat. No. 5,858,515 assigned to Kimberly-Clark Worldwide, Inc.

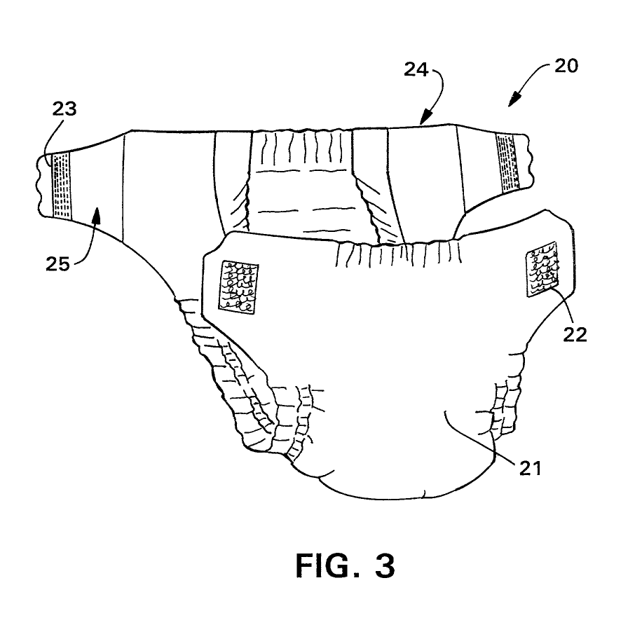

FIG. 3 shows a typical personal care product, in this case a disposable diaper 20, which has a front 21 and back 24. The front 21 has thereon a loop component 22 in the “landing area” or area of attachment for the hook component 23 which is on an “ear” 25 which is attached to, or an integral part of, the back 24. A “disposable diaper” is a particular personal care product intended and scaled to be worn by an infant. Incorporated by reference, some examples of disposable diapers are described in U.S. Pat. No. 5,151,092, U.S. Pat. No. 5,242,436 and U.S. Pat. No. 3,860,003, all of which are hereby expressly incorporated by reference.

Hooks are desirably formed of a stiffer, resilient material so that the wearer or caregiver can easily grasp the hook to disengage the fastening system and so that the hooks will spring open and recover without failure. This resiliency is what that enables the hook to resume its curved configuration after being bent as a result, for example, of the disengagement of the mating interlocking materials.

The term resilient refers not only to the hook but also to the backing material and the backing structure. The backing material is that material to which the base of the hook is attached and which is usually the same material from which the hook is formed. The backing structure refers to the combination of the backing material of the hook and/or loop and any underlying material, as well as to its configuration.

Previous investigators have discovered that absorbent articles including an interlocking fastening component having a resilient, concave backing structure are well suited to maintain proper attachment of the fastening system and such a system is described in U.S. Pat. No. 5,722,968, incorporated herein by reference in its entirety. This patent teaches that the edges and corners of the resilient, contoured interlocking fastening components do not project tangentially away from the body of the wearer as is the case with conventional flat interlocking fastening components and result in the edges and corners of the contoured interlocking fastening components penetrating deeply into the mating interlocking materials. This deep penetration ensures that clothing or other materials passing over the garment fastening system are less likely to snag on protruding edges and corners. This is thought to be a particularly significant problem for small individuals, where the angles of curvature of the body may be greater than for less slim individuals.

The inventors have surprisingly found that a fastening component shaped opposite to that taught in U.S. Pat. No. 5,722,968, i.e. one that is convex, provides superior resistance to peel forces.

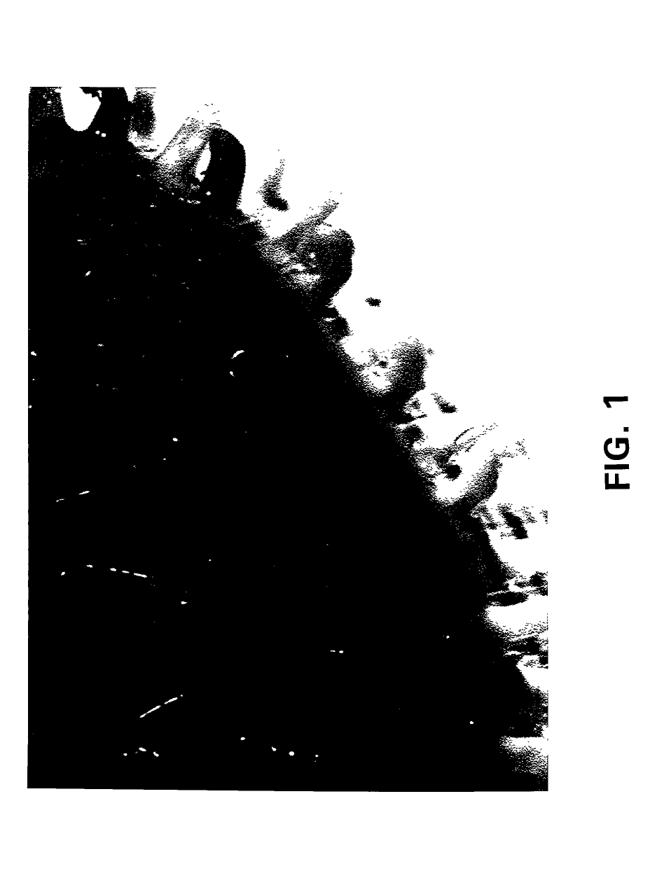

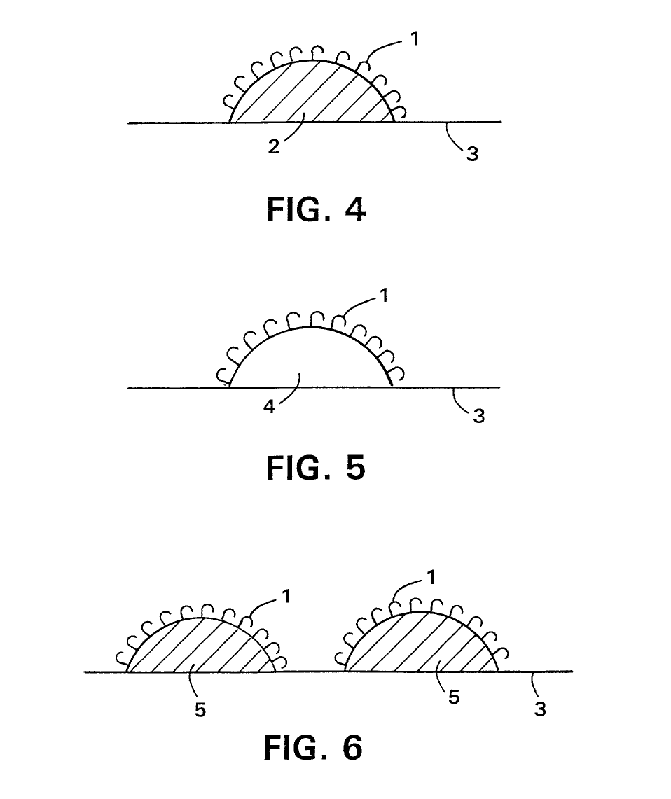

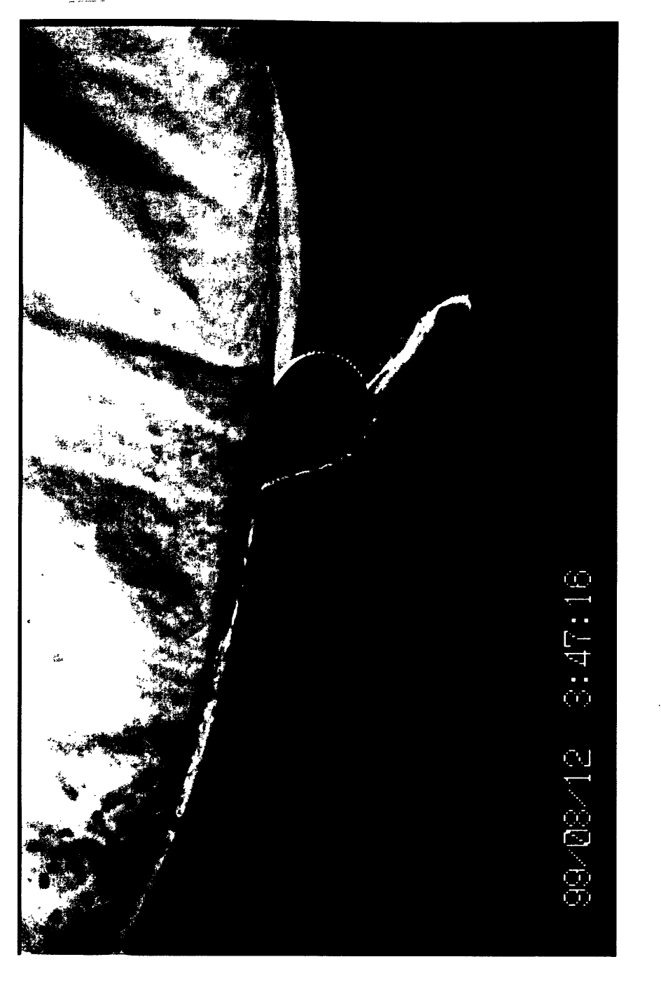

The convexity of the instant invention is clearly visible in the photograph, which is FIG. 1, which in this case shows the individual hooks and the clear curvature of the hook component. FIG. 4 is a cross sectional view of a hook component 1 which is convex and which is underlain by a resilient material 2 which is in turn connected in some manner to a backing 3. The backing 3 may be, for example, the ear as shown in FIG. 3. The embodiment having a resilient material under the hooks may also be seen in FIG. 8 where the hooks are attached to the loops on both sides of the resilient material, and in FIG. 9 where the hook component is partially unconnected from the loop component.

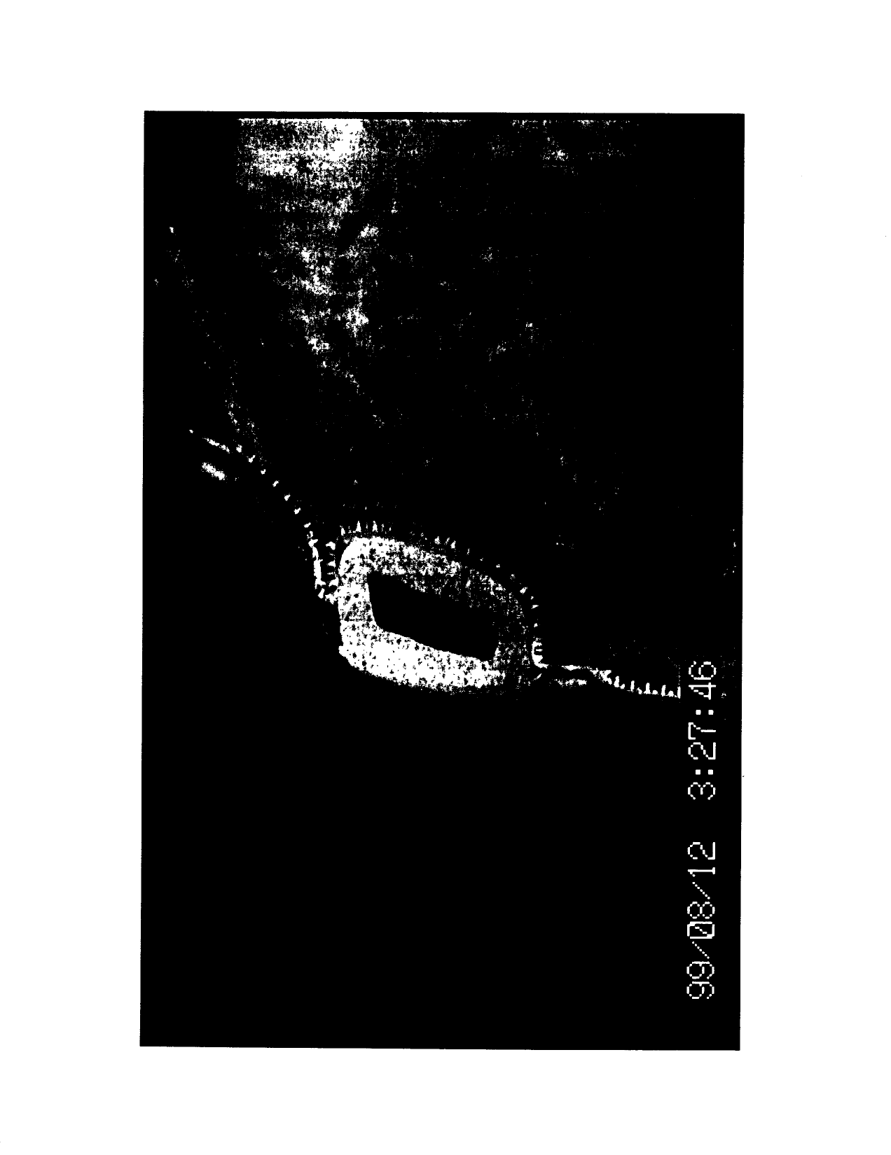

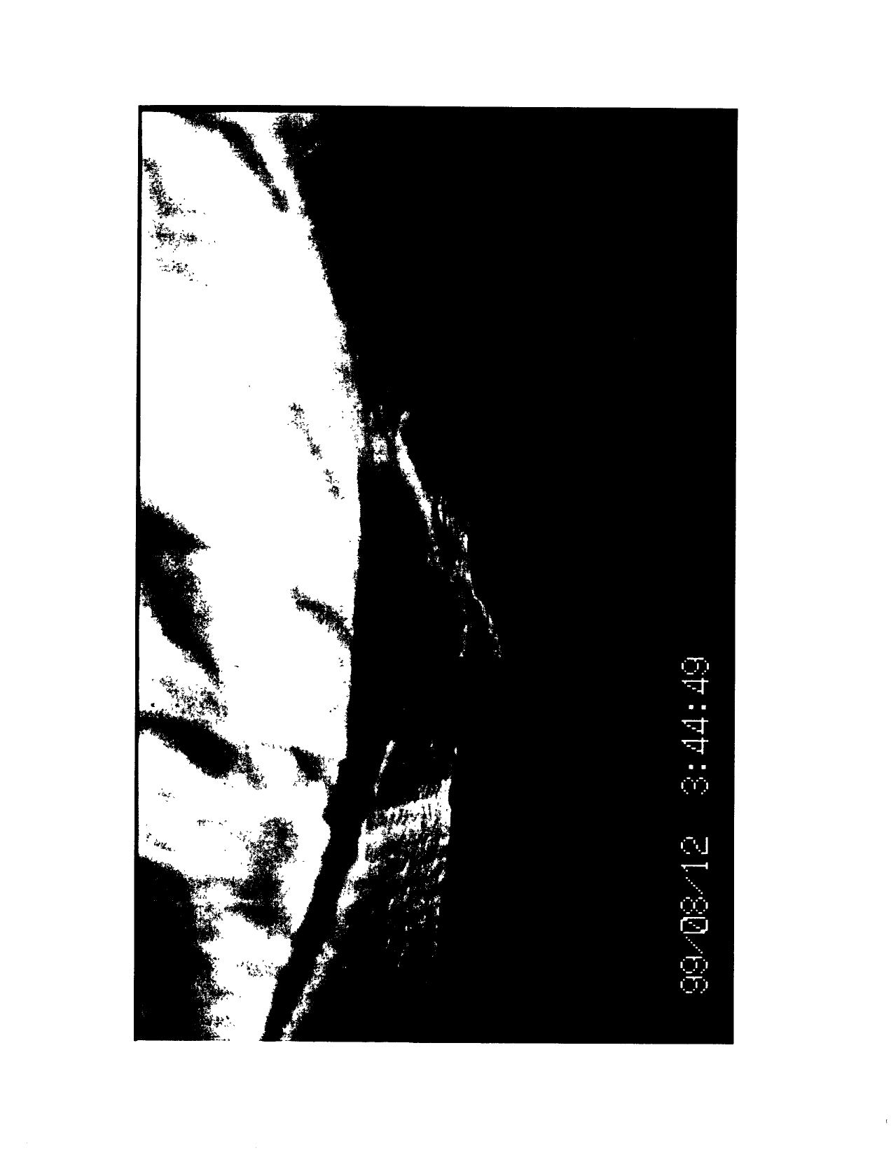

FIG. 5 shows a hook component 1 which is folded or pleated in such a manner as to rise out of the flat surface of the backing 3 without the requirement of a resilient underlayment. In the configuration of FIG. 5, a hollow or void area 4 underlies the hook component 1. Another embodiment having a hollow or void area under the hooks is visible in the photograph that is FIG. 7. FIG. 6 is a variation of FIG. 4 wherein multiple convexities are formed in the hook component 1 over an underlayment 5 which may be a resilient material or hollow, in the same manner as single convexities are formed.

As noted previously, the hook component may be placed on the ears, straps or body of a personal care product. The loops, therefore, may also be on either the ears, straps or body of the personal care product. In an alternative embodiment, the loops may be underlain by a resilient material in a manner similar to that shown in FIG. 4, for example, while the hooks may be underlain by a relatively flat material. The loop material may also alternatively be pleated so that multiple convexities are formed and so the loops are underlain by void or hollow areas in a similar manner to that shown in FIG. 5. The stiffness of the loops may be controlled by the amplitude and frequency of the pleats.

The ability of the resilient interlocking fastening component to retain its configuration can be enhanced by controlling the construction of the backing structure. The backing structure of the interlocking fastening component is desirably formed of a construction or a material that has sufficient memory characteristics such that the backing structure will return approximately to a its original configuration after repeated engagement and disengagement cycles with a complementary fastening component. Because the resilient interlocking fastening components are desirably incorporated on attachment straps or ears, the resilient interlocking fastening components should maintain their curvature in order to remain effective during use. For reasons of comfort and performance, the absorbent articles are generally constructed of flexible components so that they conform to the body of the wearer. Additionally, the loop of the fastening system, which either forms an integral part of the garment or is a separate element attached to the garment, desirably comprises a generally flexible material. Consequently, the loop tends to conform to the shape of the wearer and enhance comfort, however, this invention is not meant to be limited to embodiments in which only the loop material is that which is nearest the body of a wearer.

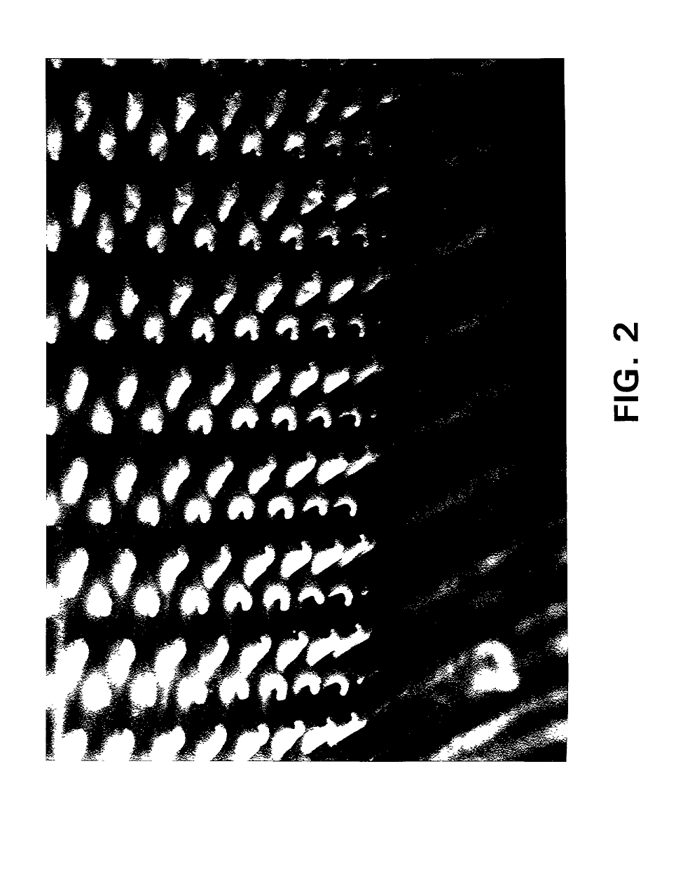

The hook component, comprising many individual hooks or “prongs”, may have such individual hooks aligned in various direction, i.e. there is no prohibition against hooks facing in different directions. FIG. 2, in fact, shows hooks pointed in two directions. The individual hooks can be arranged in the machine direction, the cross direction, or any angle between the two. While the theoretical arrangement of individual hooks is almost infinite, practically they are made in only a small number of ways; all in one direction or half in each of two directions. It should be noted that these particularities of individual hook placement are dependent on the end use and objective of the investigator and all cannot possibly be recited here. They are, however, within the understanding and ability of those skilled in the art without undue experimentation. The instant invention effects hook configuration because the movement of the resilient backing structure results in the movement of the hooks toward and away from each other as the resilient backing structure responds to the movement of the wearer and the shear forces on the personal care product.

In order to determine the amount of increase in the strength due to the instant invention, various configurations were tested according to the peel and shear tests described in the Test Methods section above. These configurations were (1) a flat, control; (2) a round sponge rubber core; (3) a “D” shaped sponge rubber core; and (4) a double “D” shaped sponge rubber core. The results are given in the Table below.

TABLE

| Material | Peak load peel, gm/2 inch width | Peak load shear, gm/in² |
|---|---|---|
| 1 | 445 | 2115 |
| 2 | 566 | 2388 |
| 3 | 575 | 3025 |
| 4 | 546 | 3384 |

The inventors have found, as can been seen from the results in the Table, that a hook and loop fastener in which at least one component is convex with a resilient material or hollow area underlaying it has at least a 20 percent superior peel strength to that of a similar hook material without a resilient material or hollow underlayment. In addition, the shear strength is also superior and could be 50 percent greater or more. The values in the Table also indicate that a more resilient material, like numbers 3 and 4, give a greater increase in shear strength than a relatively less resilient material such as that of number 2. The strength of hook and loop systems according to the invention is also superior to that of mechanical fasteners using concave hook fasteners. The inventors believe that the source of such superiority must be the rounded orientation of the hook and loop fastener material. Its believed that when the hook and loop fastening system is placed under peel forces, the components engage each other even more strongly as one component rolls upward in response to the imposition of the peeling force.

The peel and shear forces peak during testing as the materials are pulled apart. After the peak, the tensile force need to pull the pieces apart falls off, eventually to zero when the pieces are completely separated. In embodiments with more than one convexity, however, the force tends to fall after a peak corresponding to the first convexity and then rise for the second convexity, again reaching a peak and falling off. Placing a multiplicity of convexities under a component of the hook and loop system results in a multiplicity of peaks in the force needed to pull the materials apart. Appropriately sizing and spacing these convexities can result in a more uniform force need to separate the portions since the valleys between the peaks can be minimized.

As will be appreciated by those skilled in the art, changes and variations to the invention are considered to be within the ability of those skilled in the art. Such changes and variations are intended by the inventors to be within the scope of the invention.

It should further be noted that any patents, applications or publications referred to herein are incorporated by reference in their entirety.

What is claimed is:

1) A hook and loop attachment means comprising a hook portion and a loop portion wherein at least one of said portions has at least one convex portion.

2) The hook and loop attachment means of claim 1 wherein said convex portion is underlain by a resilient material.

3) A hook and loop attachment means of claim 1 wherein said convex portion is underlain by avoid.

4) The hook and loop attachment means of claim 1 wherein said attachment means has a peak peel strength at least 20 percent greater than an attachment means lacking a convex portion.

5) The hook and loop attachment means of claim 1 having two convex portions.

6) A personal care product comprising the hook and loop attachment means of claim 1.

7) The personal care product of claim 6 which is a diaper.

8) The personal care product of claim 6 which is a training pant.

9) The personal care product of claim 6 which is an incontinence product.

10) The personal care product of claim 6 which is a bandage.

11) The personal care product of claim 6 which is a feminine hygiene product.

12) The hook and loop attachment means of claim 3 wherein said hollow area is formed by pleating.

13) The hook and loop attachment means of claim 3 wherein said attachment means has a peak peel strength at least 20 percent greater than an attachment means lacking a convex portion.

14) The hook and loop attachment means of claim 3 having two convex portions.

15) A personal care product comprising the hoop and loop attachment means of claim 3.

16) The personal care product of claim 15 which is a diaper.

17) The personal care product of claim 15 which is a training pant.

18) The personal care product of claim 15 which is an incontinence product.

19) The personal care product of claim 15 which is a bandage.

20) The personal care product of claim 15 which is a feminine hygiene product.

21) A personal care product comprising a hook and loop attachment means wherein said attachment means has a peak peel strength at least 20 percent greater, and a peak shear strength at least 50 percent greater, than an attachment means lacking a convex portion.
